(19) United States
(12) Patent Application Publication
(10) Pub. No.: US 2014/0276007 A1
(43) Pub. Date: Sep. 18, 2014

(54) Surgical Pointer Having Constant Pressure

(71) Applicant: Synaptive Medical (Barbados) Inc., Bridgetown (BB)

(72) Inventors: Gal Sela, Toronto (CA); Cameron Piron, Toronto (CA); Joshua Richmond, Toronto (CA)

(21) Appl. No.: 14/215,780

(22) Filed: Mar. 17, 2014

Related U.S. Application Data
(60) Provisional application No. 61/798,391, filed on Mar. 15, 2013.

(51) Int. Cl. A61B 5/06; A61B 19/00

(52) U.S. Cl. CPC A61B 5/064; A61B 19/54
USPC 600/424; 600/431

(57) ABSTRACT

A medical navigation system having a surgical pointer is provided. The surgical pointer is used in the medical navigation system. The surgical pointer comprises an external sheath and an internal tool having a proximal end and a distal end. The internal tool is contained at least partially within the external sheath. The internal tool has a tip at the distal end that extends beyond the external sheath. The tip engages a surface of a patient. The internal tool further has a tracking marker trackable by the navigation system. The surgical pointer further has a linking component contained at least partially within the external sheath and interfacing with the internal tool. The linking component is configured to control pressure exerted by the tip on the surface of the patient.

### CROSS REFERENCE TO RELATED APPLICATIONS

The present application claims priority to U.S. Provisional Application No. 61/798,391, filed Mar. 15, 2013, the entirely of which is hereby incorporated by reference.

### FIELD

The present disclosure generally relates to surgical tools and equipment, and more specifically, to a surgical pointer having a constant pressure of application for use in minimally invasive therapy and image guided medical procedures.

### BACKGROUND

Medical imaging systems often make use of fiducial markers in the field of view of the location to be imaged in order to act as references to locate point correspondences between multiple images, or between images and the physical environments. In image guided medical procedures, fiducial markers may be used, for example, to help identify the location of imaged internal tumors or organs with respect to a patient's external anatomy. In other instances, fiducial markers can be used for comparing various imaging outputs against one another, or against previously acquired scans of a patient's anatomy.

During surgery, fiducial pointers, in the form of wands or handheld pointers, are used to identify fiducial points on a patient's surface.

Referring to FIG. 1A, an exemplary fiducial pointer tool 102 is illustrated, similar to pointer tools produced by Scopis GmbH. The fiducial pointer tool 102 may be considered an exemplary instrument for navigation having either a straight or slightly blunt tip 103. The slenderness of the tip 103 on a handheld pointer allows for precise positioning and localization of external fiducial markers on the patient.

In some instances, fiducial pointers are objects contacting the patient's body, while in other instances, fiducial pointers can be used to draw fiducial markings on a patient's skin or to trace a particular surface contour of a patient's body.

Referring to FIG. 1B, the use of a fiducial pointer on a patient's body is illustrated. In FIG. 1B, an operator 105, typically a nurse or surgeon, would use the fiducial pointer 102 to identify the location of fiducial markers 104 on a patient's face in order to register the location of the patient's anatomy in the navigation system.

However, the readings of current fiducial pointers have inherent deficiencies that affect imaging correspondence and the accuracy of localized surgery guided by these readings. In particular, the application of excessive or unknown pressure to the skin by the user of the fiducial pointer 102 can deform the skin resulting in registration of a point that is displaced relative to the true location on the surface of the skin. This displacement can be as high as a centimeter depending on the mechanics of the skin surface and the underlying tissue layer. Errors in registering the location of a fiducial point can translate into inaccuracies in the surgical procedure, since these points are used to cross reference medical images, which may be obtained through various modalities, to a localization system for navigated surgery.

Further, in some instances, deformations made by a sharp tip may puncture a patient's skin, resulting in bleeding or scarring. Thus, there exists a need to provide more accurate and sensitive fiducial pointing tools.

### SUMMARY

This disclosure describes a surgical pointer that allows for accurately drawing or selecting points on a patient's surface, for example, for defining the patient's surface for navigation registration. The apparatus, system, and methods described herein provide fiducial markers that exhibit consistent position readings, regardless of pressure changes applied by the user of the pointer, or by different users of the pointer. As a result, the present apparatus, system and methods can provide more accurate and repeatable representations of the tip location on the surface of the skin than representations generated by the standard rigid pointer tool.

One aspect of the present description provides a surgical pointer. The surgical pointer comprises an external sheath and an internal tool having a proximal end and a distal end. The internal tool is contained at least partially within the external sheath. The internal tool has a tip at the distal end that extends beyond the external sheath. The tip engages a surface of a patient. The internal tool further has a tracking marker trackable by the navigation system. The surgical pointer further has a linking component contained at least partially within the external sheath and interfacing with the internal tool. The linking component is configured to control pressure exerted by the tip on the surface of the patient.

Another aspect of the present description provides a medical navigation system having a surgical pointer and a controller. The surgical pointer comprises an external sheath and an internal tool having a proximal end and a distal end. The internal tool is contained at least partially within the external sheath. The internal tool has a tip at the distal end that extends beyond the external sheath. The tip engages a surface of a patient. The internal tool further has a tracking marker trackable by the navigation system. The surgical pointer further has a linking component contained at least partially within the external sheath and interfacing with the internal tool. The linking component is configured to control pressure exerted by the tip on the surface of the patient. The linking component includes a spring, a pneumatic cylinder, and/or an electric motor. The controller at least electrically couples to the surgical pointer. The surgical pointer transmits data to the controller. The data may indicate actuation of the tip to the medical navigation system in order to assist in image-guided surgery.

A further understanding of various aspects of the subject matter can be realized by reference to the following detailed description and drawings.

### BRIEF DESCRIPTION OF THE DRAWINGS

In order that the subject matter may be readily understood, embodiments are illustrated by way of examples in the accompanying drawings, in which:

FIG. 1A is an exemplary fiducial pointing tool;

FIG. 1B is an exemplary use of a fiducial pointer on a patient's body;

FIG. 2 is a block diagram illustrating components of a medical navigation system that may be used in conjunction with a surgical pointer for a minimally invasive surgical procedure;

FIG. 3A is a flow chart illustrating a method involved in a surgical procedure using the navigation system of FIG. 2;

FIG. 3B is a flow chart illustrating a method of registering a patient for a surgical procedure as outlined in FIG. 3A;

FIG. 4A illustrates an example of a surgical pointer having a spring mechanism;

FIG. 4B illustrates an example of a surgical pointer having a pneumatic mechanism; and

FIG. 4C illustrates an example of a surgical pointer having an electric motor.

### DETAILED DESCRIPTION

Referring to FIG. 2, a block diagram is shown illustrating components of an exemplary medical navigation system 200. The medical navigation system 200 illustrates the context in which a surgical pointer, such as that described herein, may be used. The medical navigation system 200 includes one or more monitors 205, 211 for displaying a video image, an equipment tower 201, and a mechanical arm 202, which supports an optical scope 204. The equipment tower 201 is mounted on a frame (e.g., a rack or cart) and may contain a computer or controller, planning software, navigation software, a power supply and software to manage the mechanical arm 202, and tracked instruments. In one example, the equipment tower 201 may be a single tower configuration with dual display monitors 211, 205, however other configurations may also exist (e.g., dual tower, single display, etc.). Furthermore, the equipment tower 201 may also be configured with a universal power supply (UPS) to provide for emergency power, in addition to a regular AC adapter power supply.

A patient's anatomy may be held in place by a holder. For example, in a port-based neurosurgical procedure the patient's head may be held in place by a head holder 217, and an access port 206 and an introducer 210 may be inserted into the patient's head. The introducer 210 may be tracked using a tracking camera 213, which provides position information for the navigation system 200. In one example, the tracking camera 213 may be a 3D optical tracking stereo camera, similar to one made by Northern Digital Imaging (NDI), configured to locate reflective sphere tracking markers 212 in 3D space. In another example, the tracking camera 213 may be a magnetic camera, such as a field transmitter, where receiver coils are used to locate objects in 3D space, as is also known in the art. Location data of the mechanical arm 202 and access port 206 may be determined by the tracking camera 213 by detection of tracking markers 212 placed on these tools, for example the introducer 210 and associated pointing tools. The secondary display 205 may provide output of the tracking camera 213. In one example, the output may be shown in axial, sagittal and coronal views as part of a multi-view display.

As noted above with reference to FIG. 2, the introducer 210 may include tracking markers 212 for tracking. The tracking markers 212 may be reflective spheres in the case of an optical tracking system or pick-up coils in the case of an electromagnetic tracking system. The tracking markers 212 are detected by the tracking camera 213 and their respective positions are inferred by the tracking software.

One aspect of the present description provides a medical navigation system, such as the medical navigation system 200, having a surgical pointer (e.g., the pointer tool 400, as shown in FIG. 4) and a controller (e.g., as part of equipment tower 201). The surgical pointer may have an external sheath that at least partly surrounds an internal tool, and includes a linking component. The internal tool may have a tip that extends beyond a distal end of the external sheath, and a portion that remains trackable by the navigation system tracking camera 213 when such tip is pressed against the surface of a patient. The linking component interfaces with the internal tool and the linking component is configured to control the pressure exerted by the tip on the surface of the patient. The linking component may also include a spring, a pneumatic cylinder, and/or an electric motor. The controller (e.g., in equipment tower 201) at least electrically couples to the surgical pointer (e.g., the pointer tool 410 as shown in FIG. 4B, or the pointer tool 450 as shown in FIG. 4C). The surgical pointer transmits data to the controller. The data may indicate operation of the tip to the medical navigation system 200 in order to assist in image-guided surgery.

Referring to FIG. 3A, a flow chart is shown illustrating a method 300 of performing a port-based surgical procedure using a navigation system, such as the medical navigation system 200 described in relation to FIG. 2. At a first block 302, the port-based surgical plan is imported. A detailed description of the process to create and select a surgical plan is outlined in the disclosure “PLANNING, NAVIGATION AND SIMULATION SYSTEMS AND METHODS FOR MINIMALLY INVASIVE THERAPY”, a United States Patent Publication based on a United States Patent Application, which claims priority to U.S. Provisional Patent Application Ser. Nos. 61/800,155 and 61/924,993, which are both hereby incorporated by reference in their entirety.

Once the plan has been imported into the navigation system at the block 302, the patient is affixed into position using a body holding mechanism. In the present example, a head holding mechanism 217 may be used. The head position is also confirmed with the patient plan in the navigation system (block 304), which in one example may be implemented by the computer or controller forming part of the equipment tower 201.

Next, registration of the patient is initiated (block 306). The phrase “registration” or “image registration” refers to the process of transforming different sets of data into one coordinate system. Data may includes multiple photographs, data from different sensors, times, depths, or viewpoints. The process of “registration” is used in the present application for medical imaging in which images from different imaging modalities are co-registered. Registration is used in order to be able to compare or integrate the data obtained from these different modalities.

Those skilled in the relevant arts will appreciate that there are numerous registration techniques available and one or more of the techniques may be applied to the present example. Non-limiting examples include intensity-based methods that compare intensity patterns in images via correlation metrics, while feature-based methods find correspondence between image features such as points, lines, and contours. Image registration methods may also be classified according to the transformation models they use to relate the target image space to the reference image space. Another classification can be made between single-modality and multi-modality methods. Single-modality methods typically register images in the same modality acquired by the same scanner or sensor type, for example, a series of magnetic resonance (MR) images may be co-registered, while multi-modality registration methods are used to register images acquired by different scanner or sensor types, for example in magnetic resonance imaging (MRI) and positron emission tomography (PET). In the present disclosure, multi-modality registration methods may be used in medical imaging of the head and/or brain as images of a subject are frequently obtained from different scanners. Examples include registration of brain computerized tomography (CT)/MRI images or PET/CT images for tumor localization, registration of contrast-enhanced CT images against non-contrast-enhanced CT images, and registration of ultrasound and CT.

Referring now to FIG. 3B, a flow chart is shown illustrating a method involved in registration block 306 as outlined in FIG. 3A, in greater detail. If the use of fiducial touch points (340) is contemplated, the method involves first identifying fiducials on images (block 342), then touching the touch points with a tracked instrument (block 344). Next, the navigation system computes the registration to reference markers (block 346).

Alternately, registration can also be completed by conducting a surface scan procedure (block 350). The block 350 is presented to show an alternative approach, but may not typically be used when using a fiducial pointer. First, the face is scanned using a 3D scanner (block 352). Next, the face surface is extracted from MR/CT data (block 354). Finally, surfaces are matched to determine registration data points (block 356).

Upon completion of either the fiducial touch points (340) or surface scan (350) procedures, the data extracted is computed and used to confirm registration at block 308, shown in FIG. 3A.

Referring back to FIG. 3A, once registration is confirmed (block 308), the patient is draped (block 310). Typically, draping involves covering the patient and surrounding areas with a sterile barrier to create and maintain a sterile field during the surgical procedure. The purpose of draping is to eliminate the passage of microorganisms (e.g., bacteria) between non-sterile and sterile areas.

Upon completion of draping (block 310), the patient engagement points are confirmed (block 312) and then the craniotomy is prepared and planned (block 314).

Upon completion of the preparation and planning of the craniotomy (block 314), the craniotomy is cut and a bone flap is temporarily removed from the skull to access the brain (block 316). Registration data is updated with the navigation system at this point (block 322).

Next, the engagement within craniotomy and the motion range are confirmed (block 318). Next, the procedure advances to cutting the dura at the engagement points and identifying the sulcus (block 320).

Thereafter, the cannulation process is initiated (block 324). Cannulation involves inserting a port into the brain, typically along a sulci path as identified at 320, along a trajectory plan. Cannulation is typically an iterative process that involves repeating the steps of aligning the port on engagement and setting the planned trajectory (block 332) and then cannulating to the target depth (block 334) until the complete trajectory plan is executed (block 324).

Once cannulation is complete, the surgeon then performs resection (block 326) to remove part of the brain and/or tumor of interest. The surgeon then decannulates (block 328) by removing the port and any tracking instruments from the brain. Finally, the surgeon closes the dura and completes the craniotomy (block 330). Some aspects of FIG. 3 are specific to port-based surgery, such portions of blocks 328, 320, and 334, but the appropriate portions of these blocks may be skipped or suitably modified when performing non-port based surgery.

Referring to FIG. 4A, an example of a surgical pointer 400 using a spring mechanism is illustrated. In one example, the surgical pointer 400 may be a fiducial pointing tool. In FIG. 4A, the surgical pointer 400 includes an external sheath 402 and an internal tool 404. The external sheath 402 has a proximal end (e.g., towards the top side of FIG. 4A) and a distal end (e.g., towards the bottom side of FIG. 4A) forming a handle for a user (e.g., a surgeon or operator) to grasp. The internal tool 404 may be elongated and have an axis through its length, and the internal tool 404 may be at least partly contained within external sheath 402. The internal tool 404 also includes a tip 408 which extends beyond the distal end of the external sheath 402, for contact with a patient or other surface areas. In one example, the internal tool 404 may be rigidly attached to tracked markers 212 that are trackable by a navigation system, such as the medical navigation system 200. The tip 408 of the internal tool 404 engages a patient's surface (e.g., a patient's face) for tracing or selecting fiducial marker points.

As shown in FIG. 4A, part of the interface between the external sheath 402 and the internal tool 404 may include a linking component, which controls the pressure exerted by the internal tool 404 when contacting the subject or other surface areas at the tip 408. The exemplary linking component shown in FIG. 4A shows a constant force spring 406 that may be configured to provide a substantially constant force along an axis of the internal tool 404 as internal tool 404 is pressed against the subject or other surface areas. In a further example, the constant force spring 406 may be configured to provide a resistive force along the axis of the internal tool 406 that causes minimal patient skin (or other surface) deflection. The constant force spring 406 is shown mounted on spindle 410, however, those skilled in the art will appreciate that other means for mounting a constant force spring are possible.

During operation, the user holds the external sheath 402 and pushes the tip 408 of the internal tool 404 onto the surface of the patient or registration object. If force being applied by the tip 408 is greater than a threshold determined by the design of the spring 406, the spring 406 retreats from its equilibrium or rest position and external sheath 402 will slide toward the patient by an amount not to exceed a limit of applied force, according to the design and calibration of the constant force spring 406.

In FIG. 4A, the spring 406 is shown attached to or interfacing with the proximal end of the internal tool 404. However, in other examples, the spring 406 may be attached to the distal end of the internal tool 404, or anywhere between the distal end and the proximal end of the internal tool 404, depending on design requirements. The internal tool 404 is free to move linearly along its axis, and in one example, may have a set length of travel within the external sheath 402. In some embodiments, the apparatus, system and methods of the present application can include means for signaling that excessive pressure of the pointing device against a surface has been detected. For example, if the surgical pointer 400 used to locate a point on a subject's body is pressed against the body with too much force, such that the skin may deflect unacceptably, the apparatus, system and methods herein can provide feedback to the user to inform the user of this. In some examples, audio, visual, or tactile feedback can be provided to signal excessive pressure related to tissue deformation however persons of skill will appreciate that the apparatus, system and methods can be implemented by means of any feedback mechanism capable of providing notification to the user. In one example, if internal tool 404 is moved toward an unacceptable range for spring 406, the internal tool 404 may be blocked by a stop (not shown) in order to protect the integrity of constant force spring 406 and/or an alarm or other means of alerting of such out-of-range operation may also be triggered.

In another example, a dial or sliding gauge may be mounted on the external sheath 402 and attached to or coupled with the internal tool 404 to give the user feedback as to the amount of compliance available or when the force limit is in jeopardy of being exceeded.

In yet another example, the surgical pointer 450 may optionally be coupled to a controller, as described in more detail below in connection with FIG. 4C, in order to provide data to the controller, such as the medical navigation system 200.

In an exemplary embodiment, tip 408 may contain a force sensor that may provide a signal to the controller (as shown in FIG. 4C at 458). It will be apparent to those of skill in the art that in such an example, the resistive force of constant force spring 406 must be higher than the force sensitivity threshold of the tip sensor for the tip sensor to be effective.

Referring to FIG. 4B, another exemplary surgical pointer 410 is shown. While different references numerals are used for the exemplary surgical pointers shown in FIGS. 4A-4C, it should be understood that many aspects of the surgical pointers shown in FIGS. 4A-4C are similar and features described in connection with any of FIGS. 4A-4C may be equally applicable to the surgical pointers shown in the other FIGS. 4A-4C.

The surgical pointer 410 shown in FIG. 4B may be pneumatically operated. In FIG. 4B, the surgical pointer 410 has an external sheath 412. An internal tool 414 having a tip 418 at the distal end of the internal tool 414 is mounted within the external sheath 412. The user also holds the external sheath 412 and contacts the patient surface with the tip 418 of the internal tool 414. The internal tool 414 may be free to move along its axis within the external sheath 412. The internal tool 414 may be rigidly attached to tracked markers 212 that are trackable by a navigation system, such as the medical navigation system 200.

As shown in FIG. 4B, the internal tool 414 is connected to or coupled with a pneumatic cylinder 416, which may be configured to apply a load to the internal tool 414 such that the force is directed along the axis of the internal tool 414 toward the distal end of the internal tool 414 such that pressure applied to the surface of the patient by the tip 418 is substantially constant.

The pneumatic cylinder 416 may have a pressure relief valve 420 that connects the pneumatic cylinder 416 to an external air supply source, indicated by reference 422. In another example, the external air supply source 422 may be attached to the external sheath 412 or may be internal to the surgical pointer 410.

The pressure relief valve 420 may have either a set or variable pressure limit that may be triggered when the force applied to the tip of the internal tool 414 exceeds a certain threshold limit. In one example, the pressure relief valve 420 releases air such that the pressure and, as a result the force applied to the internal tool 414, the tip 418 remains at the limit.

In another example, the user may hold the surgical pointer 410 away from the surface of the patient and control the pneumatic cylinder 416 to drive the tip 418 of internal tool 414 towards the patient surface. When the tip 418 contacts the patient surface with the force set by the limit of the pressure relief valve 420, the advancement of internal tool 414 will be halted. Alternatively, a hydraulic cylinder and valve may be used as a replacement for pneumatic cylinder 416, but may be similarly configured as shown in FIG. 4B.

The surgical pointer 410 may be coupled to a controller, as described in more detail below in connection with FIG. 4C, in order to provide data to the controller, such as can be found in the medical navigation system 200. The controller may further control or regulate the air supply source 422, according to the design criteria of a particular application. In one example, the controller may provide active control of the internal tool 414, typically via an electric motor or pneumatic cylinder. A sensor on the tip, such as the tip 418, may relay the applied force to the controller. The controller may actuate the pneumatics or electric motor to affect the applied force, for example in a closed-loop feedback system.

Referring to FIG. 4C, an example of a surgical pointer 450 using an electric motor is shown. In FIG. 4C, the surgical pointer 450 has an external sheath 452. Mounted within the external sheath 452 is an internal tool 454 having a tip 456 at the distal end of the internal tool 454. The internal tool 454 may be rigidly attached to tracking markers 212 that are trackable by a navigation system, such as the medical navigation system 200. In one example, an electric motor 462 is attached to the proximal end of the internal tool 454. The electric motor 462 may also be attached to the external sheath 452. In one example, the electric motor 462 may be a servo motor. However, any suitable motor may be used and may be located in any suitable location relative to the internal tool 454, according to the design criteria of a particular application.

The electric motor 462 may attach to the internal tool 454 via a lead screw or other suitable mechanism that translates the electric motor 462 angular motion to a linear motion resulting in linear actuation of the internal tool 454. This combination of the electric motor 462 and linear mechanism may be replaced by any other suitable electrical linear actuator known to persons skilled in the relevant arts.

A force sensor 458 may be attached to the tip 456 of the internal tool 454 and the force sensor 458 may be used to generate a signal representing a measurement of the force applied to the patient surface. A signal (e.g., a data signal) representing the measurement may be communicated to a controller 464. The force sensor 458 may be equally applicable to the tips 418 and 408 shown in FIGS. 4A and 4B, respectively.

Feedback may be provided by the controller 464, for example on a display (e.g., displays 205, 211). While the controller 464 is shown in connection with FIG. 4C, the controller may be equally applicable to the surgical pointers shown in FIGS. 4B and 4A. For example, if the surgical pointer 450 (or surgical pointers 400, 410) is pressed against the body with too much force, such that the skin may deflect and/or alter the point's coordinates, the surgical pointer 450 may provide feedback to the user to inform the user of this deflection. As an example, the force sensor 458 may provide a signal to the controller 464 indicating the amount of force being exerted on the tip 456. The controller 464 may provide the feedback to the user, in various ways. In some examples, audio (e.g., through a speaker coupled to the controller 464 such as can be found in the medical navigation system 200), visual (e.g., from a display coupled to the controller 464, such as displays 205, 211) or tactile feedback (e.g., using a feedback mechanism designed into the surgical pointer 450, such as a vibrator or any other suitable mechanism) may be provided to signal excessive pressure related to tissue deformation. It will be appreciated that feedback can be implemented using other mechanisms capable of providing a notification to the user. For example, feedback could be communicated via an apparatus or accessory on the surgical pointer 450 itself. This feedback could be advantageous, for example, in systems with low tolerance for tissue deflection, that use fiducial points on a patient to aid in image registration.

The controller 464 may run a control loop (e.g., a proportional-integral-derivative (PID) control) to drive the electric motor 462 with the suitable gain such that the applied force measured by force sensor 458 is substantially constant. The controller 464 may signal to the user via audio, visual, or tactile means that the force limit has been approached, reached, or exceeded.

The examples illustrated in connection with FIGS. 4B and 4C may support two modes of operation, which include: (a) relief of excessive force applied by the user, and (b) actively driving the tip 418, 456 of the internal tool 414, 454 towards the patient surface until the measured force indicates that the specified or desired force has been met.

Further in FIG. 4C, a laser projector 460 may project a dot of light onto the patient surface at the location to which the tip 456 of the internal tool 454 is to be driven. The laser projector 460 provides targeting assistance for the user, aimed at the eventual contact point. When the user is in position, the surgical pointer 450 may advance the internal tool 454 until contact is made at the specified pressure.

The location and orientation of the laser projector 460 with respect to the internal tool 454 as shown in FIG. 4C, is one example only. Any suitable location, orientation, and/or configuration of the laser projector 460 relative to the internal tool 454 may be used. The laser projector 460 may also be considered an add-on accessory that is also applicable to other exemplary embodiments, in particular, to a tool that is capable of actively driving the tip of the internal tool to contact the patient's surface.

In some examples, control signals may be sent by the surgical pointer 450 when the pressure is above or below a specified range, to trigger additional outputs other than the feedback mechanisms disclosed above. In some examples, the surgical pointer 450 may switch to an altered state, or stop surface data collection, if a pressure above or below a given threshold is detected. In the examples presented in FIG. 4B and FIG. 4C, the pressure relief valve 420 or the force sensor 458 may be used to sense that the pressure limit has been achieved or exceeded.

In some examples, the linking component may combine some or all of a spring, fluid filled chambers, servo motors, or cushioning materials. Persons of skill in the relevant arts will appreciate that any suitable pressure regulating mechanism may be utilized, according to the design criteria of a particular application.

In use, the examples described herein may be used for pre-operative surgical planning, intra-operative surgical navigation, and post-operative education review of a procedure, such as for a surgeon self-assessment or student education and review, retrospective determination of deformation of points of interest for subsequent imaging, and follow-up assessment. In use, the examples described herein tend to allow for accurate readings of fiducial points or fiducial contouring of surfaces, by ensuring that fiducial points remain fixed regardless of the pressure exerted when contacting the surface.

At least some of the elements of the systems described herein may be implemented by software, or a combination of software and hardware. Elements of the system that are implemented via software may be written in a high-level procedural language such as object oriented programming or a scripting language. Accordingly, the program code may be written in C, C++, J++, or any other suitable programming language and may comprise modules or classes, as is known to those skilled in object oriented programming. At least some of the elements of the system that are implemented via software may be written in assembly language, machine language or firmware as needed. In either case, the program code can be stored on storage media or on a computer readable medium that is readable by a general or special purpose programmable computing device having a processor, an operating system and the associated hardware and software that is necessary to implement the functionality of at least one of the embodiments described herein. The program code, when read by the computing device, configures the computing device to operate in a new, specific and predefined manner in order to perform at least one of the methods described herein.

Furthermore, at least some of the methods described herein are capable of being distributed in a computer program product comprising a computer readable medium that bears computer usable instructions for execution by one or more processors, to perform aspects of the methods described. The medium may be provided in various forms such as, but not limited to, one or more diskettes, compact disks, DVDs, tapes, chips, USB keys, external hard drives, wire-line transmissions, satellite transmissions, internet transmissions or downloads, magnetic and electronic storage media, digital and analog signals, and the like. The computer useable instructions may also be in various forms, including compiled and non-compiled code.

While the teachings described herein are in conjunction with various embodiments for illustrative purposes, it is not intended that the teachings be limited to such embodiments. On the contrary, the teachings described and illustrated herein encompass various alternatives, modifications, and equivalents, without departing from the described embodiments, the general scope of which is defined in the appended claims. Except to the extent necessary or inherent in the processes themselves, no particular order to steps or stages of methods or processes described in this disclosure is, intended or implied. In many cases the order of process steps may be varied without changing the purpose, effect, or import of the methods described.

What is claimed is:

1. A surgical pointer for use in a navigation system, the surgical pointer comprising:
an external sheath;
an internal tool having a proximal end and a distal end, the internal tool contained at least partially within the external sheath, the internal tool having a tip at the distal end that extends beyond the external sheath, the tip for engaging a surface of a patient, the internal tool further having a tracking marker trackable by the navigation system; and
a linking component contained at least partially within the external sheath and interfacing with the internal tool, the linking component configured to control pressure exerted by the tip on the surface of the patient.

2. The surgical pointer according to claim 1, wherein the linking component includes a spring.

3. The surgical pointer according to claim 1, wherein the linking component includes a pneumatic cylinder.

4. The surgical pointer according to claim 1, wherein the linking component includes an electric motor.

5. The surgical pointer according to claims 1, wherein the linking component provides a substantially constant force to the internal tool in an axial direction of the internal tool toward the distal end such that pressure applied to the surface of the patient by the tip is substantially constant.

6. The surgical pointer according to claim 4, wherein the electric motor includes a servo motor coupled to a mechanism, the mechanism coupled to the internal tool, the mechanism translating angular motion of the electric motor to a linear motion resulting in linear actuation of the internal tool.

7. The surgical pointer according to claim 1, wherein the internal tool is free to move in a linear fashion in an axial direction of the internal tool and the internal tool has a set length of travel within the external sheath such that the external sheath operates as a handle that may be held such that the tip of the internal tool is positioned against the surface of the patient.

8. The surgical pointer according to claim 3, further comprising a pressure relief valve coupled to the pneumatic cylinder for connecting the pneumatic cylinder to an air supply source, the pressure relief valve having a pressure limit that is triggered when the force applied to the tip exceeds a threshold.

9. The surgical pointer according to claim 1, further comprising a force sensor attached to the tip.

10. The surgical pointer according to claim 9, wherein the force sensor generates a signal representing the force applied to the surface of the patient, the signal for use by a controller of the navigation system electrically coupled to the surgical pointer.

11. The surgical pointer according to claim 1, wherein the internal tool has two or more of the tracking markers and the tracking markers extend beyond the external sheath.

12. The surgical pointer according to claim 11, wherein the tracking markers are attached to the proximal end of the internal tool, the proximal end of the internal tool extending beyond the external sheath.

13. A medical navigation system, comprising:
a surgical pointer having:
an external sheath;
an internal tool having a proximal end and a distal end, the internal tool contained at least partially within the external sheath, the internal tool having a tip at the distal end that extends beyond the external sheath, the tip for engaging a surface of a patient, the internal tool further having a tracking marker; and
a linking component contained at least partially within the external sheath and interfacing with the internal tool, the linking component configured to control pressure exerted by the tip on the surface of the patient; and
a controller at least electrically coupled to the surgical pointer, the surgical pointer transmitting data to the controller, the data indicating operation of the tip to the medical navigation system in order to assist in image-guided surgery.

14. The medical navigation system according to claim 13, wherein the linking component includes at least one of: a spring, a pneumatic cylinder, and an electric motor.

15. The medical navigation system according to claim 13, wherein the linking component provides a substantially constant force to the internal tool in an axial direction of the internal tool toward the distal end such that pressure applied to the surface of the patient by the tip is substantially constant and wherein control of the linking component is effected by the controller.

16. The medical navigation system according to claim 13, further comprising a force sensor attached to the tip, wherein the force sensor generates a signal representing the force applied to the surface of the patient, the signal being communicated to the controller.

17. The medical navigation system according to claim 14, wherein:
when the linking component includes the pneumatic cylinder, the controller controls a source of air pressure coupled to the pneumatic cylinder to provide a substantially constant force to the internal tool in an axial direction of the internal tool such that pressure applied to the surface of the patient by the tip is substantially constant; and
when the linking component includes the electric motor, the electric motor includes a servo motor and the controller controls the servo motor to provide a substantially constant force to the internal tool in an axial direction of the internal tool such that pressure applied to the surface of the patient by the tip is substantially constant.

18. The medical navigation system according to claim 13, wherein the internal tool has two or more of the tracking markers and the tracking markers extend beyond the external sheath.

19. The medical navigation system according to claim 18, wherein the tracking markers are attached to the proximal end of the internal tool, the proximal end of the internal tool extending beyond the external sheath.
